(19) United States
(12) Patent Application Publication
(10) Pub. No.: US 2023/0038615 A1
(43) Pub. Date: Feb. 9, 2023

(54) MEDICAL DEVICES AND RELATED SYSTEMS AND METHODS

(71) Applicant: Orthosensor Inc., Dania Beach, FL (US)

(72) Inventor: Joseph DeCerce, Fort Lauderdale, FL (US)

(73) Assignee: Orthosensor Inc., Dania Beach, FL (US)

(21) Appl. No.: 17/880,156

(22) Filed: Aug. 3, 2022

Related U.S. Application Data
(60) Provisional application No. 63/229,352, filed on Aug. 4, 2021.

(51) Int. Cl. A61B 5/00

(52) U.S. Cl. CPC A61B 5/4585; A61B 5/742; A61B 5/6847; A61B 2562/0261; A61B 2562/166; A61B 2562/046; A61B 5/748

(57) ABSTRACT

A medical device may include a lower housing, a printed circuit board (PCB) received within the lower housing, a top housing, two plungers received in gaps within the top housing, and strain gauges mounted to the PCB. The strain gauges may define two polygons aligned with the two plungers. The two plungers may be in contact with the strain gauges such that a force on a plunger is transferred to the strain gauges in contact with the respective plunger. The load magnitude and load location may be determined based on the measured strain of the strain gauges.

### CROSS-REFERENCE TO RELATED APPLICATIONS

This application claims the benefit of priority under 35 U.S.C. § 119 from U.S. Provisional Application No. 63/229,352, filed on Aug. 4, 2021, which is incorporated by reference herein in its entirety.

### TECHNICAL FIELD

The present disclosure relates generally to medical devices, and more particularly to medical devices including one or more strain gauges.

### BACKGROUND

The skeletal system of a mammal is subject to variations among species. Further changes can occur due to environmental factors, degradation through use, and aging. An orthopedic joint of the skeletal system typically comprises two or more bones that move in relation to one another. Movement is enabled by muscle tissue and tendons attached to the skeletal system of the joint. Ligaments hold and stabilize the one or more joint bones positionally. Cartilage is a wear surface that prevents bone-to-bone contact, distributes load, and lowers friction.

There has been substantial growth in the repair of the human skeletal system. In general, orthopedic joints have evolved using information from simulations, mechanical prototypes, and patient data that is collected and used to initiate improved designs. Similarly, the tools being used for orthopedic surgery have been refined over the years but have not changed substantially. Thus, the basic procedure for replacement of an orthopedic joint has been standardized to meet the general needs of a wide distribution of the population.

Although the tools, procedures, and artificial joints meet a general need, each replacement procedure is subject to significant variation from patient to patient. The correction of these individual variations relies on the skill of the surgeon to adapt and fit the replacement joint using the available tools to the specific circumstance. The gathering of relevant data on parameters within the joint may assist the surgeon in replacing the joint. The solution of this disclosure may resolve these and/or other issues of the art.

### SUMMARY

In one aspect, a medical device may include a lower housing and a circuit board within the lower housing. A first plurality of strain gauge sensor may be mounted to a medial portion of the circuit board, and a second plurality of strain gauge sensors may be mounted to a lateral portion the circuit board. An upper housing may couple to the lower housing. The device may also include a first plunger received by the upper housing that is in contact with each of the first plurality of strain gauge sensors and a second plunger received by the upper housing that is in contact with each of the second plurality of strain gauge sensors.

In other aspects, the medical device may include one or more of the following features. The first plunger may comprise a first plurality of bridge portions, the second plunger may comprise a second plurality of bridge portions. Each bridge portion of the first plurality of bridge portions may be in contact with a strain gauge sensor of the first plurality of strain gauge sensors, and each bridge portion of the second plurality of bridge portions may be in contact with a strain gauge sensor of the second plurality of strain gauge sensors. The first plunger may be configured such that a first force applied to a top side of the first plunger is applied across the first plurality of strain gauge sensors, and the second plunger may be configured such that a second force applied to a top side of the second plunger is applied across the second plurality of strain gauge sensors.

The first plurality of strain gauge sensors may be positioned to define a medial polygon wherein vertices of the medial polygon are at center points of each of the first plurality of strain gauge sensors, and the second plurality of strain gauge sensors may be positioned to define a lateral polygon wherein vertices of the lateral polygon are at center points of each of the second plurality of strain gauges sensors. The first plurality of strain gauge sensors may include a first strain gauge sensor mounted at a first mounting area, a second strain gauge sensor mounted at a second mounting area, and a third strain gauge sensor mounted at a third mounting area, and the first mounting area, the second mounting area, and the third mounting area may be positioned around the perimeter of the medial portion of the circuit board and protrude outwardly from a center portion of the medial portion. The second plurality of strain gauge sensors may include a fourth strain gauge sensor mounted at a fourth mounting area, a fifth strain gauge sensor mounted at a fifth mounting area, and a sixth strain gauge sensor mounted at a sixth mounting area, and the fourth mounting area, the fifth mounting area, and the sixth mounting area may be positioned around the perimeter of the lateral portion of the circuit board and protrude outwardly from a center portion of the lateral portion.

The first strain gauge sensor may be mounted such that a first reference axis of the strain gauge sensor forms an angle of 0-15 degrees with a first central axis of the longitudinal axis of the medial portion, and the first reference axis extends perpendicular to a second central longitudinal axis of the first strain gauge sensor and across a first width of the first strain gauge sensor. The second strain gauge sensor may be mounted such that a second reference axis of the second strain gauge sensor forms an angle of 0-40 degrees with the first central longitudinal axis, wherein the second reference axis extends perpendicular to a third central longitudinal axis of the second strain gauge sensor and across a second width of the second strain gauge sensor. The third strain gauge sensor may be mounted such that a third reference axis of the third strain gauge sensor forms an angle of 0-90 degrees with the first central longitudinal axis, wherein the third reference axis extends perpendicular to a fourth central longitudinal axis of the third strain gauge sensor and across a third width of the third strain gauge sensor.

The first strain gauge sensor, the second strain gauge sensor, and the third strain gauge sensor may be angled relative to each other. The first plunger may include a first o-ring configured to form a seal with the upper housing, and the second plunger may include a second o-ring configured to form a seal with the upper housing. Each strain gauge sensor of the first plurality of strain gauge sensors may be coupled to a separate mounting portion of the first plurality of mounting portions, and each strain gauge sensor of the second plurality of strain gauge sensors may be coupled to a separate mounting portion of the second plurality of mounting portions. The first plurality of strain gauge sensors and the second plurality of strain gauge sensor may be mounted to the circuit board with springs. Each of the first plurality of strain gauge sensors and each of the second plurality of strain gauge sensors may include a plurality of strain sensors.

In another aspect, a method for determining a load magnitude and location at a joint using a medical device may include measuring strain at a first plurality of strain gauge sensors and a second plurality of strain gauge sensors. The method may also include determining at least one load magnitude and at least one load location based on the measured strain. The medical device used in the method may include a lower housing and a circuit board within the lower housing. The first plurality of strain gauge sensors may be mounted to a medial portion of the circuit board, and a second plurality of strain gauge sensors may be mounted to a lateral portion of the circuit board. An upper housing may couple to the lower housing. The device may also include a first plunger received by the upper housing that is adjacent to the first plurality of strain gauge sensors and a second plunger received by the upper housing that is adjacent to the second plurality of strain gauge sensors.

Another aspect of the method for determining a load magnitude and location at a joint using a medical device may include one or more of the following features. The first plunger may comprise a first plurality of bridge portions, the second plunger may comprise a second plurality of bridge portions. Each bridge portion of the first plurality of bridge portions may be in contact with a strain gauge sensor of the first plurality of strain gauge sensors, and each bridge portion of the second plurality of bridge portions may be in contact with a strain gauge sensor of the second plurality of strain gauge sensors. The first plurality of strain gauge sensors may be positioned to define a medial polygon wherein vertices of the medial polygon are at center points of each of the first plurality of strain gauge sensors, and the second plurality of strain gauge sensors may be positioned to define a lateral polygon wherein vertices of the lateral polygon are at center points of each of the second plurality of strain gauges sensors.

The method may further include measuring a first load applied to the first plunger by measuring a first strain on each of the strain gauge sensors that define the medial polygon and measuring a second load applied to the second plunger by measuring a second strain on each of the strain gauge sensors that define the lateral polygon. A medial load magnitude and a medial load location on the first plunger may be determined from the first strain, and a lateral load magnitude and lateral load location on the second plunger may be determined from the second strain. The medial load magnitude, medial load location, lateral load magnitude, and lateral load location may be displayed on a graphical user interface (GUI).

In yet another aspect, a medical device may include a lower housing and a circuit board within the lower housing with a medial portion and a lateral portion. A first plurality of strain gauge sensors with at least three strain gauge sensors may be mounted to the medial portion, and a second plurality of strain gauge sensors with at least three strain gauge sensors may be mounted to the lateral portion. An upper housing with a lateral lumen and a medial lumen may be coupled to the lower housing. The device may also include a medial plunger configured to be received in the medial lumen and a lateral plunger configured to be received in the lateral lumen. The first plurality of strain gauge sensors may be in contact with a first surface of the medial plunger and the second plurality of strain gauge sensors may be in contact with a second surface of the lateral plunger. The first plurality of strain gauge sensors and the second plurality of strain gauge sensors may be mounted to the circuit board via springs.

In some aspects, the medical device may include one or more of the following features. The medial plunger may include a first plurality of bridge portions and the lateral plunger may include a second plurality of bridge portions. Each bridge portion of the first plurality of bridge portions may be in contact with a strain gauge sensor of the first plurality of strain gauge sensors, and each bridge portion of the second plurality of bridge portion may be in contact with a strain gauge sensor of the second plurality of strain gauge sensors. The medial plunger may be configured to distribute force applied to a top side of the medial plunger across the first plurality of strain gauge sensors, and the lateral plunger is configured to distribute force applied to a top side of the lateral plunger across the second plurality of strain gauge sensors.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 shows an isometric view of a medical implant, according to aspects of this disclosure;

FIG. 2 shows an exploded view of the medical implant of FIG. 1, according to aspects of this disclosure;

FIG. 3 shows a lower housing of the medical implant of FIG. 1, according to aspects of this disclosure;

FIG. 4 shows a bottom view of a printed circuit board (“PCB”) that may be used in the medical implant of FIG. 1, according to aspects of this disclosure;

FIG. 5 shows a top view of a printed circuit board (“PCB”) that may be used in the medical implant of FIG. 1, according to aspects of this disclosure;

FIG. 6 shows a bottom view of a strain gauge sensor that may be used in the medical implant of FIG. 1, according to aspects of this disclosure;

FIG. 7 shows a top view of a strain gauge sensor that may be used in the medical implant of FIG. 1, according to aspects of this disclosure;

FIG. 8 shows an isometric view of the PCB of FIGS. 5 and 6 and sensors received in the lower housing of FIG. 3, according to aspects of this disclosure;

FIG. 9 shows a top view of a portion of an alternative flexible PCB including where the sensors of FIGS. 6 and 7 may be mounted, according to aspects of this disclosure;

FIG. 10 shows a top view of a plunger used in the medical implant in FIG. 1, according to aspects of this disclosure;

FIG. 11 shows a bottom view of the plunger of FIG. 9, according to aspects of this disclosure;

FIG. 12 shows an isometric view of the upper housing of the medical implant in FIG. 1, according to aspects of this disclosure;

FIG. 13 shows a cross-section of the medical implant in FIG. 1, according to aspects of this disclosure; and

FIG. 14 shows a perspective, partial cross-sectional view of a sensor mounted using springs to the PCB of FIGS. 5 and 6, according to aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Moreover, in this disclosure, relative terms, such as, for example, “about,” “substantially,” and “approximately” are used to indicate a possible variation of ±10% in the stated value. Unless stated otherwise, the term “exemplary” is used in the sense of “example” rather than “ideal.”

Embodiments of this disclosure are generally directed to medical devices for placement inside or on the surface of a body, such as the human body. Several embodiments of this disclosure are generally directed to implantable devices for use in a joint, and in particular, for use in a knee joint. Embodiments of this disclosure may also be directed to systems, medical devices, or methods using such implantable medical devices.

Although exemplary embodiments of this disclosure will be described with reference to an implant, it will be appreciated that aspects of this disclosure have wide application, and thus may be suitable for use with many types of medical devices and systems, such as a system comprising a medical implant and a remote control unit or other computer system. Accordingly, the following descriptions and illustrations should be considered illustrative in nature, and thus, not limiting the scope of this disclosure.

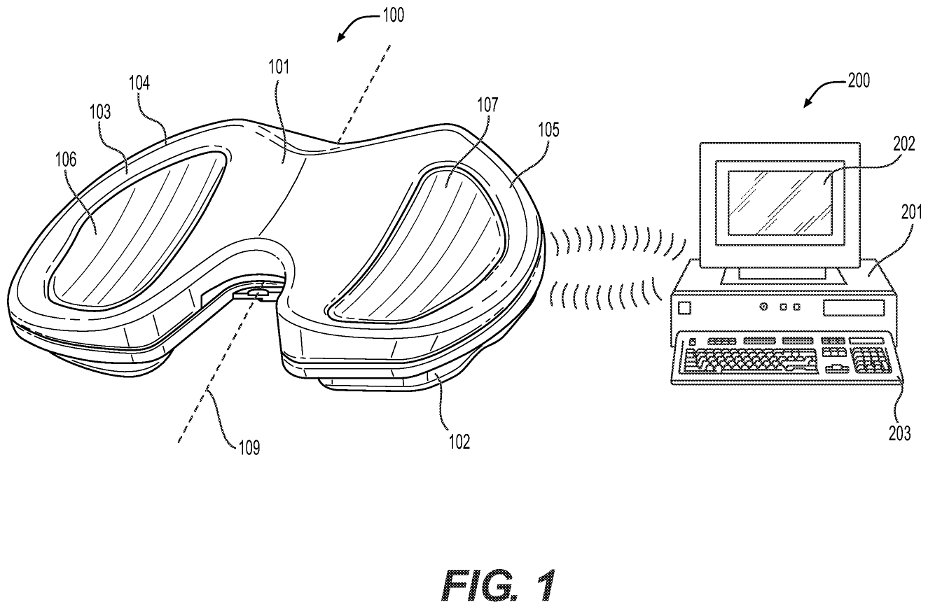

FIG. 1 illustrates a medical device or implant 100 that may be connected with a user interface on a remote system 200. The remote system 200 may be a computer, smart phone, laptop, or any similar computing device. The remote system 200 may include a control unit 201 including computer processing components, a keyboard or other input device 203 (e.g. a mouse, remote control, etc.), and an electronic display 202. The medical implant 100 may be a permanent implant, a temporary implant, a trial insert, a prosthetic component, or similar medical device. The implant 100 may be placed inside a patient's musculo-skeletal system, such as between two bones, in a patient's joint, and/or proximate to a portion of the musculo-skeletal system. The implant 100 may be placed within a patient's knee, such as between the tibia and femur, and may be coupled to one or more of the tibia and femur. The implant 100 collects data using one or more sensors. The sensors may collect data on the load placed on the implant, the range of motion of the joint, the alignment of the joint, and/or other parameters known in the art. This data may then be transmitted to the external user interface, such as a computer with a display or a portable electronic device, which may provide information to surgeons or other medical personnel. In some examples, the surgeons may make adjustments during surgery such as where cuts are made in the bone and how bones are aligned based on information provided by implant 100.

The implant 100 may include a lower housing portion 102 and an upper housing portion 104. The lower housing portion 102 and the upper housing portion 104 couple together to form the implant 100. The lower housing portion 102 and the upper housing portion 104 may be hermetically sealed together. The implant 100 has a lateral portion 103 to the left of central axis 109, a medial portion 105 to the right of central axis 109, and a joining portion 101 along central axis 109 that connects the lateral portion 103 and medial portion 105. Each of the lateral portion 103 and medial portion 105 contains a plunger 106, 107. Each plunger 106, 107 may be compressed and/or move in response to a force, and may move further into the upper housing 104. As discussed with more detail below, the compression of a plunger 106, 107 may impact and/or apply a force to one or more strain sensors 110-115 within the implant 100, which is then used to measure load magnitude, location, stability, and/or other parameters.

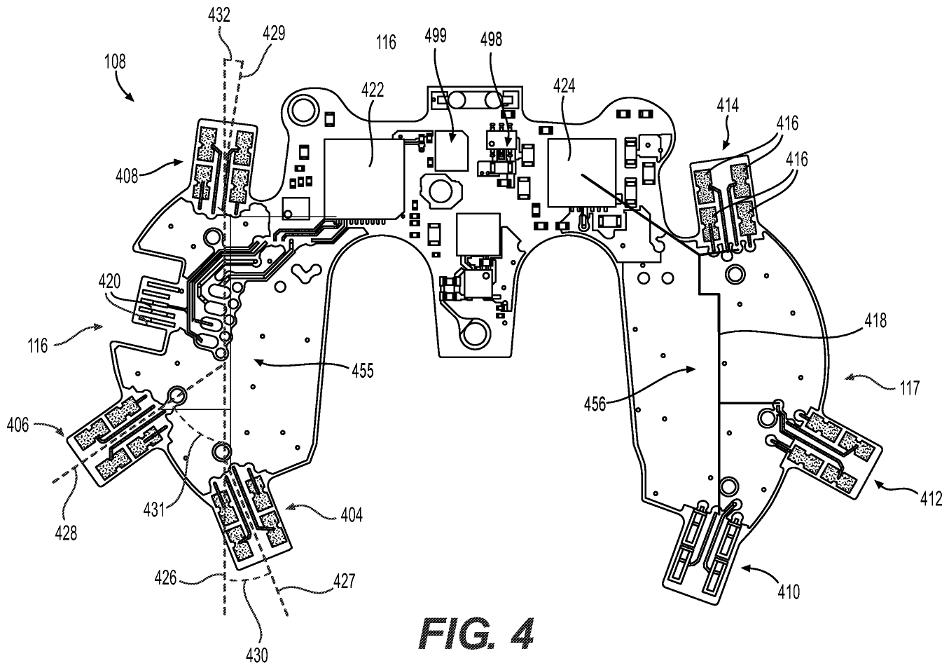

The implant 100 may contain a printed circuit board 108 (“PCB”), and PCB 108 may be configured to be received within the lower housing portion 102 and the upper housing portion 104. The PCB 108 may have a lateral PCB portion 116, a medial PCB portion 117, and a joining PCB portion 118. The PCB 108 may contain electronic components such as internal circuitry, communication circuitry, one or more power sources, one or more sensors, and other computing components. The one or more sensors mounted on the PCB 108 may include strain gauge sensors 110-115 (shown in FIG. 4). Each strain gauge sensor 110-115 may include a single strain gauge or multiple strain gauges, as described below. The strain gauge sensors 110-115 of implant 100 may be mounted on PCB 108. The strain gauge sensors 110-115 may be positioned on the PCB 108 so that when a plunger 106, 107 compresses, the strain gauge sensors 110-115 are impacted such that they are able to determine the load magnitude and location accurately.

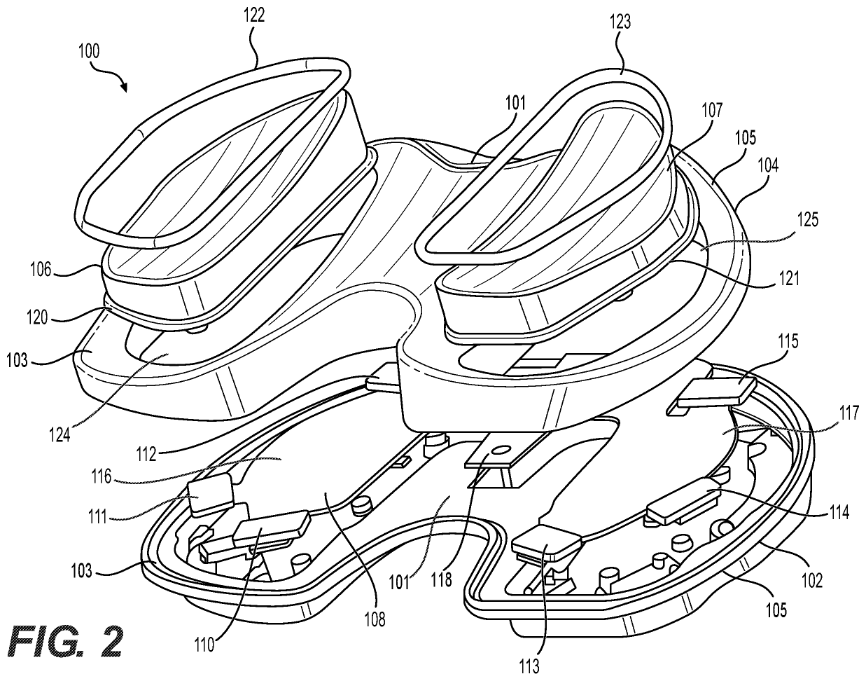

FIG. 2 illustrates an exploded view of the implant 100 of FIG. 1. As shown in FIG. 2, PCB 108 may be housed by the implant 100 in the lower housing 102. Sensors 110-115 may be mounted on the PCB 108 by, for example, using springs. In some examples, each of sensors 110-115 may be strain gauges. Sensors 110-112 may be positioned within the lateral portion 103 of implant 100 and may be coupled to the lateral PCB portion 116. Sensors 113-115 may be positioned within the medial portion 105 on the medial PCB portion 117. The relative positioning of sensors 110-115 is described with regards to FIG. 4 further below.

Two plungers 106, 107 may be placed, respectively, within lateral lumen 124 and medial lumen 125 in the upper housing 104. In one embodiment, each plunger 106, 107 may have a retaining lip 120, 121 and retaining lip 120, 121 may extend around a perimeter portion of plunger 106, 107. An O-ring 122, 123 may be placed around each plunger 106, 107 and a portion of O-ring 114 may be configured to be held in place by retaining lip 120, 121. For example, retaining lip 120, 121 may prevent movement of O-ring 122, 123 towards lower housing 102. In some examples, plunger 106 may be stopped from moving further through lumens 124, 125 of upper housing 104 when O-ring 122, 123 abuts a portion (or “lip”) of upper housing 104 extending around lumens 124, 125. O-ring 122, 123 may compress between upper housing 104 and plunger 106 to hermetically seal the lumens 124, 125 in upper housing 104. Furthermore, O-ring 122, 123 supports movement of plunger 106 while maintaining the hermetic seal during an application of a force, pressure, or load to plunger 106, 107 and strain gauges 110-115. As discussed further below, bridges 1002 may protrude outward from the bottom side 1003 of plunger 106, 107 to impact strain gauges 110-115 positioned at predetermined locations on PCB 108. The predetermined locations of the strain gauges 110-115 combined with the measurements from the strain gauges may be used to calculate the position of an applied load and/or a magnitude of a load at that predetermined position or another position on implant 100.

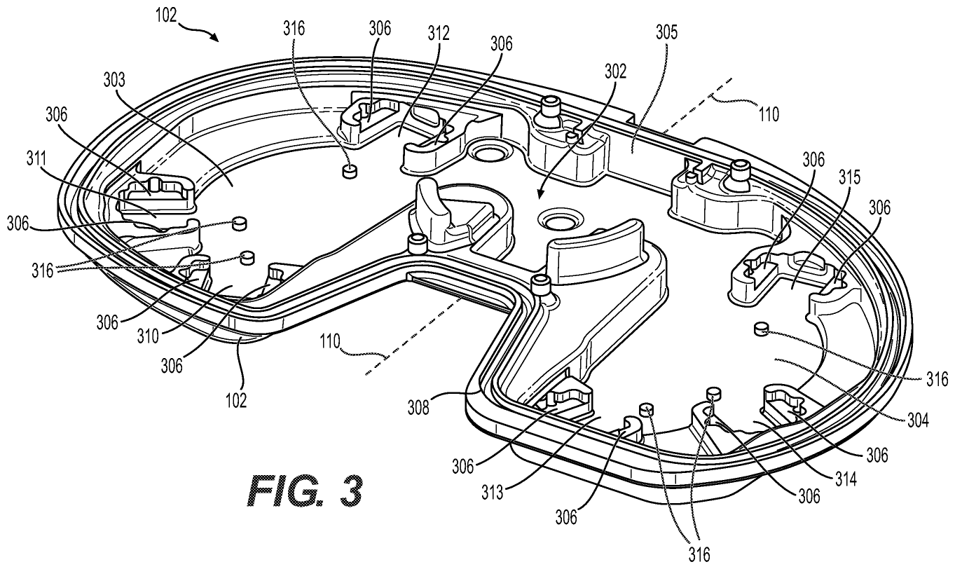

FIG. 3 shows lower housing 102 with PCB 108 and other components removed. As can be seen in FIG. 3, lower housing 102 comprises a lower housing lateral portion 303, a lower housing medial portion 304, and a lower housing joining portion 305. Each of the lower housing lateral portion 303 and medial portion 304 may include lower housing coupling components 316, such as cylindrical protrusions or other protrusions configured to couple to PCB 108, used to couple PCB 108 to lower housing 102. In some examples, lower housing 102 further comprises a PCB recess 302 configured to receive PCB 108. As shown, lower housing 102 may further comprise six strain gauge recesses 310-315 configured to receive strain gauges 110-115. In one embodiment, each strain gauge recess 310-315 may comprise two strain gauge supports 306 at opposing sides of each recess 310-315. Strain gauge supports 306 may be configured to hold opposing sides of strain gauges 110 so as to create a space between strain gauges 110-115 positioned in strain gauge recesses 310-315 and PCB 108 received in lower housing 102. This space allows strain gauges 110-115 mounted on PCB 108 to deform and measure strain without applying excessive force to PCB 108. In some examples, lower housing 102 may further comprise a receiving channel 308 around the periphery of lower housing 102 to receive upper housing 104 and to facilitate coupling of lower housing 102 and upper housing 104.

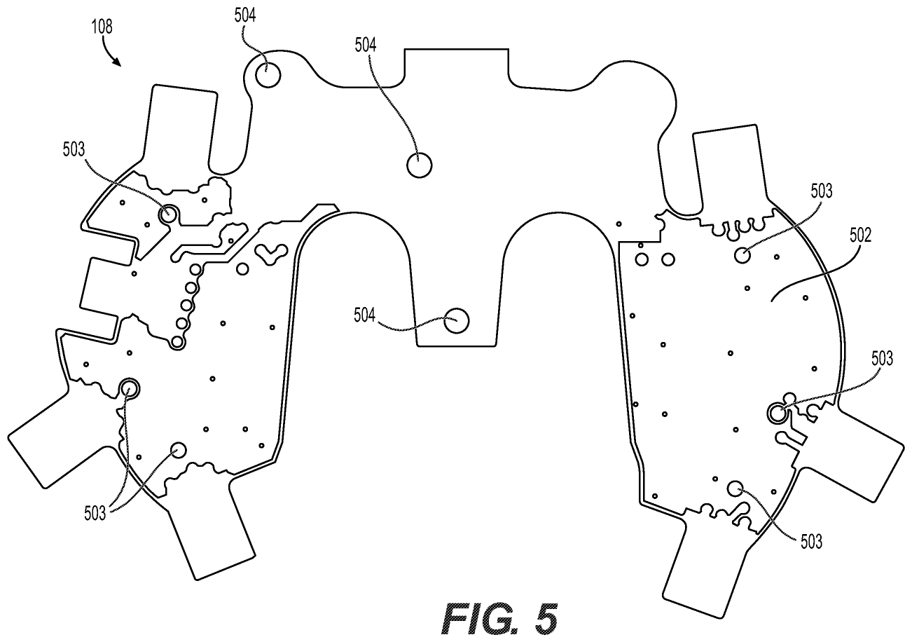

FIGS. 4 and 5 show top and bottom views, respectively, of PCB 108. PCB 108 is configured to fit into PCB recess 302. PCB 108 may include a lateral PCB portion 116, a medial PCB portion 117, and a joining PCB portion 118. Lateral PCB portion 116 and medial PCB portion 117 may be ovular with rectangular mounting portions 404-414 (see FIG. 4) protruding outwardly around their perimeter. Joining PCB portion 118 may be shaped approximately in a “T” shape. PCB 108 may be substantially planar and may include a top surface 402 and a bottom surface 502. In some examples, top surface 402 of PCB 108 may include gauge mounting areas 404, 406, 408, 410, 412, and 414. Gauge mounting areas 404-408 may be positioned around the perimeter of the lateral PCB portion 116 and may protrude outwardly from a central portion 455 of the lateral PCB portion 116. Gauge mounting areas 410-414 may be positioned around the perimeter of the medial PCB portion 117 and may protrude outwardly from a central portion 456 of the medial PCB portion 117. Gauge mounting areas may include one or more mounting portions 416. Mounting portions 416 may be where each strain gauge sensor 110-115 is coupled to PCB 108. In some examples, each strain gauge sensor 110 is coupled to PCB 108 by four springs 1304, with each spring 1304 coupled to a respective mounting portion 416 in a mounting area 404-414. The springs 1304 may be flat springs, leaf springs, coil springs, or any connector with similar elastic properties. Strain gauge sensors 110 mounted to mounting areas 404-414 may be connected to electronic components 422, 424, through PCB circuitry 418. As shown in FIG. 4, the positions of mounting areas 404, 406, 408, 410, 412, and 414 on each of the lateral PCB portion 116 and medial PCB portion 117 define a triangular polygon, and the relative positioning of mounting areas 404, 406, 408, 410, 412, and 414 may facilitate calculating the load magnitude and load location on the implant using strain gauges 110-115. For example, mounting areas 404, 406, 408 form the corners of a triangular polygon on the medial portion 116 of PCB 108, and the relative positioning of each strain gauge 110-112 mounted to each of mounting areas 404, 406, 408 facilitates the calculation of the position and magnitude of a load applied to plunger 106. The angle 430-432 of the strain gauge sensors 110-112 mounted at mounting areas 404, 406, 408 may be measured as an angle between a central longitudinal axis 426 of lateral PCB portion 116, and a central longitudinal axis 427-429 of each mounting area 404, 406, 408 extending through a center of each mounting area 404, 406, 408. In some examples, each central longitudinal axis 427-429 of each mounting area 404, 406, 408 may be parallel to the short side 701of each sensor 110-112. The angles of the strain gauge sensors 113-115 may be measured relative to a similar central longitudinal axis of medial PCB portion 117 and similar central longitudinal axes of mounting areas 410, 412, 414.

In some examples, the lateral PCB potion 116 includes first mounting area 404, second mounting area 406, and third mounting area 408. Central longitudinal axis 426 in FIG. 4 passes through the center of third mounting area 408 and is parallel to central-axis 109. First mounting area 404 may extend outward from a peripheral portion of the lateral PCB portion 116, as shown in FIG. 4. First mounting area may be offset from central longitudinal axis 426. First mounting area 404 may have a first longitudinal axis 427 evenly bisecting first mounting area 404 along its centerline. First longitudinal axis 427 may be at an acute angle 430 from central longitudinal axis 426. In some examples, angle 430 may be approximately 0-40 degrees from central longitudinal axis 426. For example, first mounting area 404 may be at an angle 430 approximately 21 degrees from vertical axis 426. Second mounting area 406 may be on the outside perimeter of lateral PCB portion 116, to the left of central longitudinal axis 426 (viewed from the top as shown in FIG. 4). Second mounting area 406 may have a second longitudinal axis 428 evenly bisecting second mounting area 406 along its centerline. Second longitudinal axis 428 may be at an angle 431 from central longitudinal axis 426, which may be at approximately 0-90 degrees. In some examples, second mounting area 406 may be at an angle 431 of 55 degrees from vertical-axis 426. Third mounting area 408 may be approximately in-line with central longitudinal axis 426. Third mounting area 408 may have a third longitudinal axis 429 evenly bisecting third mounting area 408 along its centerline. Third longitudinal axis 429 may be at an angle 432 from central longitudinal axis 426. And angle 432 may be approximately 0-15 degrees. For example, third mounting area 408 may be at an angle 432 approximately 7 degrees from central longitudinal axis 426.

The positioning of mounting areas 410, 412, 414 may mirror the positioning of mounting areas 404, 406, and 408. This configuration of mounting areas 404, 406, 408, 410, 412, 414, and specifically the positions and angles of mounting areas 404, 406, 408, 410, 412, 414, defines a triangular polygon on each of the lateral PCB portion 116 and medial PCB portion 117. In some examples, forces applied to upper housing 104 or plunger 106, 107 within these polygons may be measured by strain gauge sensors 110-115.

In some examples, PCB 108 includes circuitry and electronic components 422, 424 for processing, electronic communication, memory storage, and/or power supply. Electronic components 422, 424 may be mounted to PCB 108 at the joining PCB portion 118 or any other portion of PCB 108. Electronic components 422, 424 may include processors, digital signal processors, digital logic circuitry, interface circuitry, analog circuitry, buffers, amplifiers, radio frequency circuitry or similar communication circuitry 498, sensors, passive components, power sources, power management circuitry 499, and other circuitry. Communication circuitry 498 may be configured to send and receive signals to a remote system 200. For example, communication circuitry 498 may communicate data collected by the implant 100 to the remote system 200 or receive instructions from the remote system 200. The remote system 200 may include a display with a graphical user interface (“GUI”), and the GUI may display the information collected by implant 100 to surgeons and other medical personnel. In some examples, a portable electronic device such as a tablet or cell phone may be used as a remote system. Power sources may include at least one battery. Power management circuitry 499 may be used to control when other electronic components are supplied power, such as sensors or other components. For example, communication circuitry 498 may only be supplied power for a brief period of time, such as enough time to send a signal to remote system 200, after predetermined time intervals. In one embodiment, PCB 108 may include additional sensors, including one or more accelerometers, inertial measurement units (IMU), temperature sensors, and/or any other sensor known in the art.

FIG. 5 shows a bottom view of PCB 108. PCB 108 includes PCB outer coupling holes 503 for receiving the lower housing coupling components 316. This may secure PCB 108 to lower housing 102. PCB 108 may further include PCB inner coupling holes 504 for receiving a peg or other coupling component from the upper housing 104 to further secure PCB 108. PCB inner coupling holes 504 may be positioned on joining PCB portion 118.

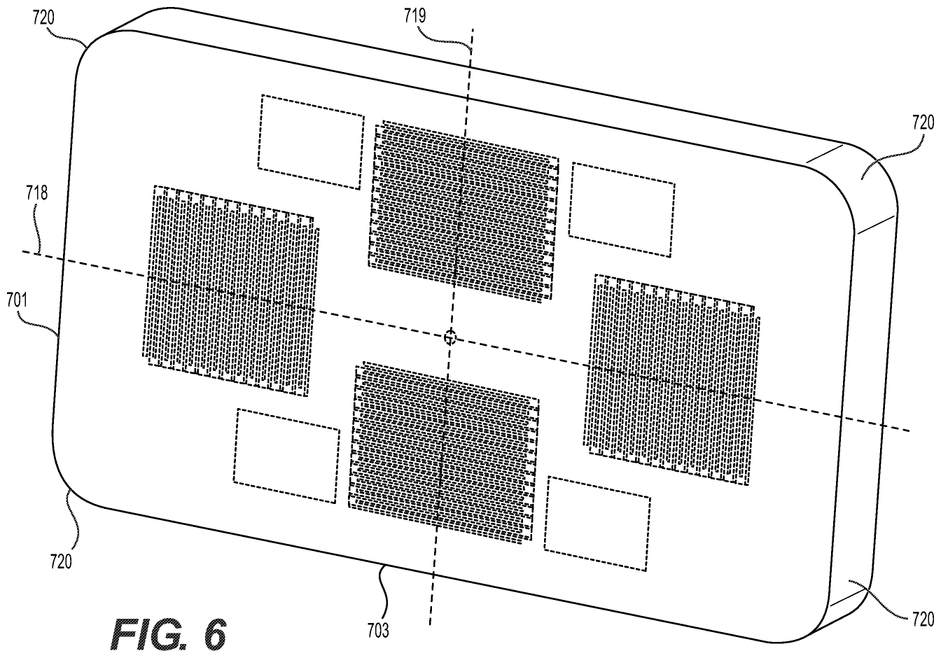

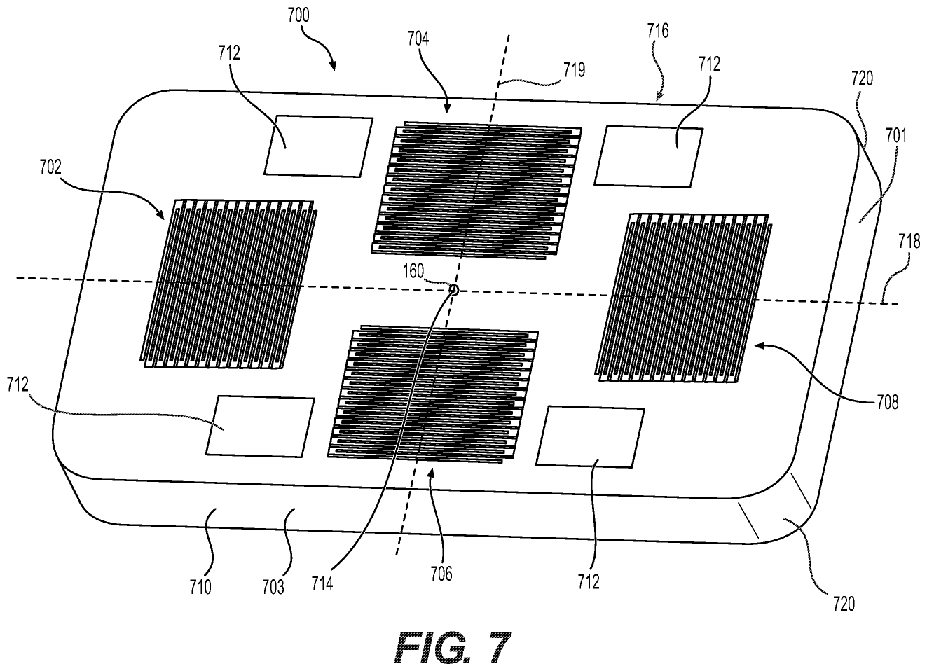

FIGS. 6-7 respectively are bottom and top views of a strain gauge sensor 700 that may be used as sensor 110. While FIG. 6 shows the top view of the strain gauge sensor 700, the shapes in dotted lines (such as the four squares in dotted lines) represent the components on the bottom side 716 of the strain gauge sensor 700. Aspects of strain gauge sensor 700 are described in U.S. patent application Ser. No. 17/158,126, filed Jan. 26, 2021, which is incorporated herein by reference in its entirety. In some examples, the strain gauge sensor 700 comprises four resistance strain gauges 702, 704, 706, 708 positioned on the bottom side 716 of a substrate 710. The substrate 710 is configured to elastically deform when a force, pressure, or load is applied. The four resistance strain gauges 702, 704, 706, and 708 may be positioned in a full bridge Poisson gauge configuration, as shown. The full bridge configuration of strain gauges 702, 704, 706, 708 may improve sensitivity, reduce noise, measurement stability, linearity of measurement, and reduce hysteresis relative to other configurations. Strain gauge sensor 700 may also comprise mounting points 712 on the bottom side 716 where strain gauge sensor 700 may be coupled to, for example, PCB 108. Mounting points 712 may be the same or a different material as substrate 710. There may be four mounting points 712 positioned at equal distances from the central longitudinal axis 718 and the reference axis 719 extending perpendicular to central longitudinal axis 718 across the width of strain gauge sensor 700. Mounting points 712 may be configured to couple directly to mounting portions 416 of the PCB 108. Mounting points 712 may also be configured to couple to mounting portions 416 of PCB 108 using springs 1304. Strain gauge sensor 700 is configured to provide accurate, repeatable, and cost efficient force, pressure, or load sensing for medical applications. In some examples, a plurality of strain gauge sensors 700 may be used to measure loading applied by a musculoskeletal system to determine load magnitude of the applied load and position of the applied load. Referring to FIG. 7, strain gauge sensor 700 may be rectangular in shape and may have rounded corner portions 720. The central longitudinal axis 718 is parallel to a long side 703 of strain gauge sensor 700. The reference axis 719 is parallel to a short side 701 of strain gauge sensor 700. Strain gauge sensor 700 has a center point 714, and in some examples strain gauge sensor 700 may be configured to measure a force, pressure, or load applied to center point 714. The angles 430-432 of the strain gauge sensors 700, such as described above in reference to FIG. 4, can be measured as between the reference axis 719 extending from the center point 714 and axis 426. The force, pressure, or load applied to strain gauge sensor 700 may be applied to an area larger than depicted by center point 714, such as along the reference axis 719 of each strain gauge sensor 700. In some examples, an origin 714 of the central longitudinal axis 718 and the reference axis 719 may correspond to center point 714. Strain gauges 704 and 706 are the active devices of strain gauge sensor 700. Strain gauge 704 is above the central longitudinal axis 718 (when viewed from the top as shown in FIG. 7), but centered on the reference axis 719. Strain gauge 706 is below the central longitudinal axis 718 (when viewed from the top as shown in FIG. 7) but centered on the reference axis 719. Note that strain gauge 704 and strain gauge 706 are configured to measure strain or deformation of substrate 710 along or parallel to the central longitudinal axis 718. In some examples, strain gauges 702 and 708 are non-active devices of strain gauge sensor 700. Strain gauge 702 is placed to the left of the reference axis 719 (when viewed from the top as shown in FIG. 7) and strain gauge 708 is placed to the right of the reference axis 719 (when viewed from the top as shown in FIG. 7). Strain gauges 704 and 706 may be positioned closer to center point 714 than strain gauges 702 and 708. Strain gauges 702 and 708 may be positioned at least a distance of one half of the length, or more, of the distance strain gauges 704 or 706 are positioned from center point 714. Strain gauges 702 and 708 are aligned to measure strain or deformation in direction of the reference axis 719. The application of a force, pressure, or load to medical implant 100 results in little or no strain to be measured by strain gauges 702, 708 such that they are considered non-active. However, strain gauges 702, 708 may still be used to normalize or correct strains measured by strain gauges 704 and 706 in order to more accurately measure strain.

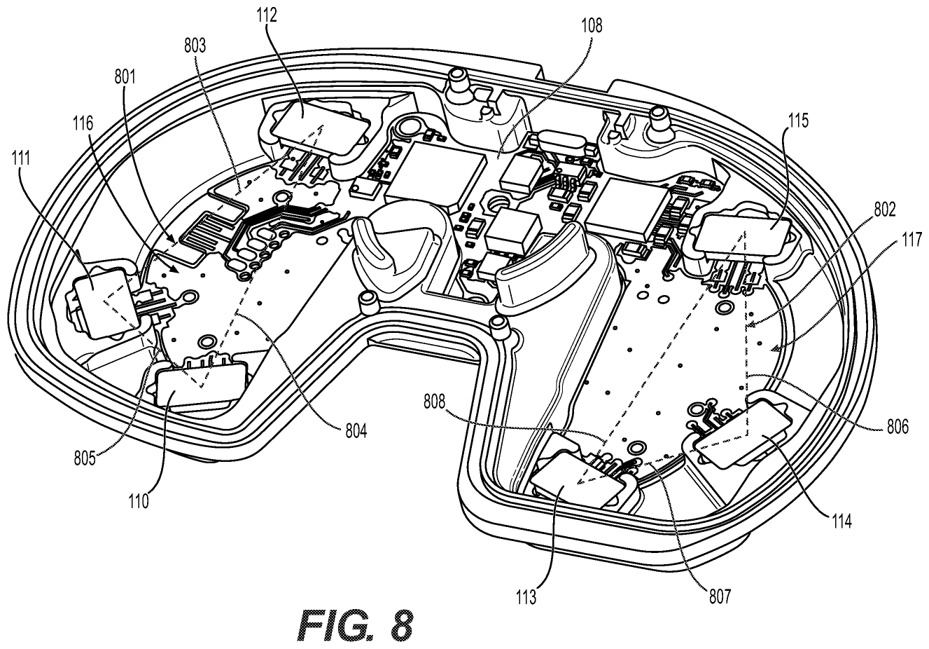

FIG. 8 shows a view of PCB 108 received in lower housing 102 with strain gauges 110 received in strain gauge recesses 310-315. In some examples, circuity 418 and electronic components 422, 424 mounted on PCB 108 may be located in joining PCB portion 118. Three strain gauge sensors 110-115 may be mounted on PCB 108 at predetermined locations on lateral and medial PCB portions 116, 117. These predetermined locations may define a polygon 801, 802 on the lateral and medial PCB portions 116, 117 between the strain gauge sensors 110-115. The polygons 801, 802 may be triangular, and each polygon 801, 802 may be positioned entirely within each of the lateral and medial PCB portions 116, 117, respectively. The polygons 801, 802 within lateral and medial PCB portions 116, 117 may be smaller than the medial and lateral portions 103, 105. As described further below, the strain applied to implant 100 may be measured at each of strain gauge sensors 110-115 that defines polygons 801, 802, and the load magnitude and location of a load on the upper housing may be accurately calculated from the measured strains at each strain gauge sensor 110-115. Each strain gauge sensor 110 may be mounted on PCB 108 using four springs 1304 to connect each of the four mounting points 712 of the strain gauges 110-115 to each mounting portion 416 on PCB 108.

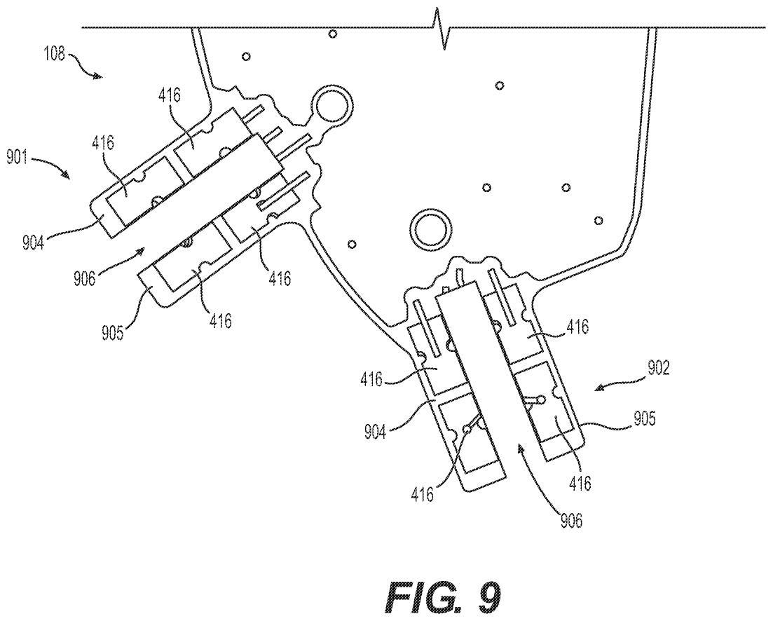

In other examples, PCB 108 may include flexible portions, such as flexible portions 901, 902 shown in FIG. 9. The flexible portions 901, 902 of PCB 108 may be created by a relief cut of a portion of PCB 108, such that two arms 904, 905 are created with the relief cut creating a gap 906 between them. For example, mounting areas 404, 406, 408, 410, 412, 414 may be bisected by a gap 906 so that mounting areas 404, 406, 408, 410, 412, 414 are all flexible, such as flexible PCB portions 901, 902. The two arms 904, 905 of PCB 108 with a gap 906 between them may move independently from one another and the flexibility of the two arms 904, 905 may prevent PCB 108 from breaking when deformed under a load. Each arm 904, 905 may have two mounting portions 416 configured to receive sensors 110-115. Sensors 110-115 may be mounted to both arms 904, 905 by coupling mounting points 712 to mounting portions 416 on each arm. In one embodiment, sensors 110-115 may be mounted so that the long side 703 of the sensor 110-115 is transverse and/or perpendicular to the direction of the relief cut. Sensor 110 may be mounted using surface mounting technology (SMT) to couple each mounting point 712 to mounting portions 416. This configuration allows for deformation of the sensor and bending of the independently moveable and flexible arms 904, 905 of PCB 108 so that sensors 110-115 may measure strain or deformation of substrate 710 along, parallel to, or otherwise transverse to, the central longitudinal axis 718 without damaging PCB 108.

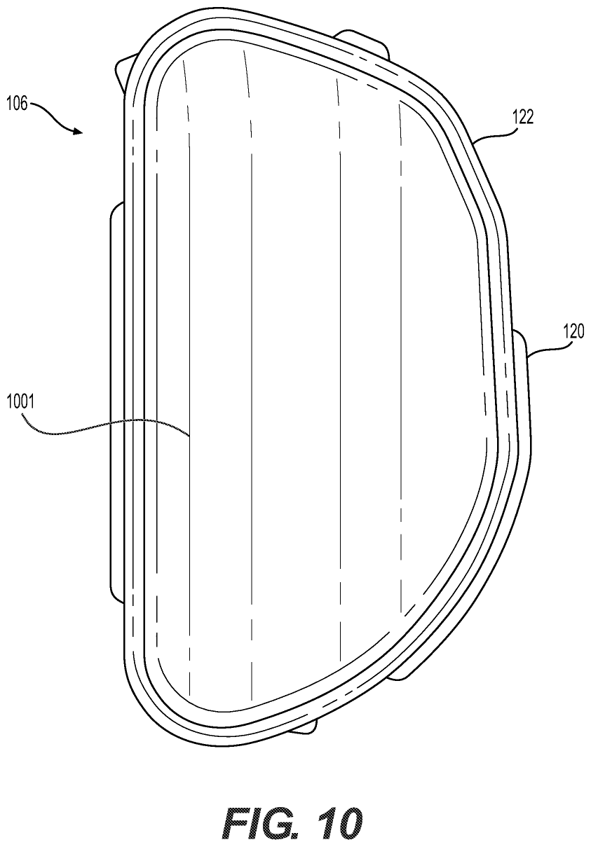

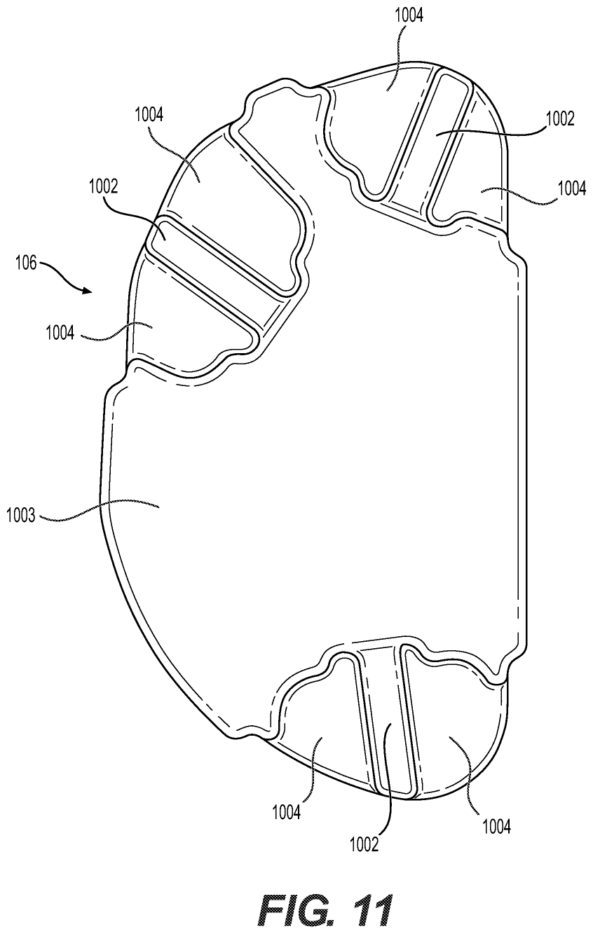

FIGS. 10-11 are respectively top and bottom views of plunger 106. Plungers 106, 107 may have a top side 1001 and a bottom side 1003. Top side 1001 and bottom side 1003 of plungers 106, 107 may be substantially semi-circular or similar in shape. Top side 1001 of plungers 106, 107 may be a concave surface. With reference to FIG. 11, plungers 106, 107 may have bridge portions 1002. Bridge portions 1002 may be designed so that when plunger 106 is under a force, bridge portions 1002 are in contact with sensors 110-115 held at predetermined locations by lower housing 102 and PCB 108 as described above. In some examples, when plunger 106, 107 is under load, the bottom of the plunger 106, 107 is stopped only by bridge portions 1002 impacting sensors 110-115. In other examples, plunger 106 abuts or is in contact with each of the three sensors 110-112 at bridge portions 1002. In either case, the contact points where bridge portions 1002 impact sensors 110-112 are the only points of contact of the plunger 106 with sensors 110-112, and therefore the load on the plunger is concentrated on sensors 110-112. In some examples, bridge portions 1002 impact sensors 110-115 that are strain gauges 700, and bridge portions 1002 are aligned with the center point 714 along the reference axis 719 of each respective strain gauge 700. When the sensors 110-115 in the lateral and medial portions 103, 105 are three strain gauge sensors 700 placed at predetermined locations that define a polygon (such as polygons 801, 802), the location and magnitude of a load on top of the plungers 106, 107 may be calculated based on the measured strains.

With reference to FIG. 11, the bottom view of plunger 106 is shown. In some examples, two plungers 106, 107 may be used in implant 100. One plunger 106 may be received by lumens 124, 125 of lateral and medial portions 103, 105 of upper housing 104. The top surface 1001 of plunger 106 may be concave. O-ring 122, 123 may be configured to be held around plunger 106, 107 by retaining lip 120, 121 located between the top 1001 and bottom surface 1003, around the perimeter of plunger 106, 107.

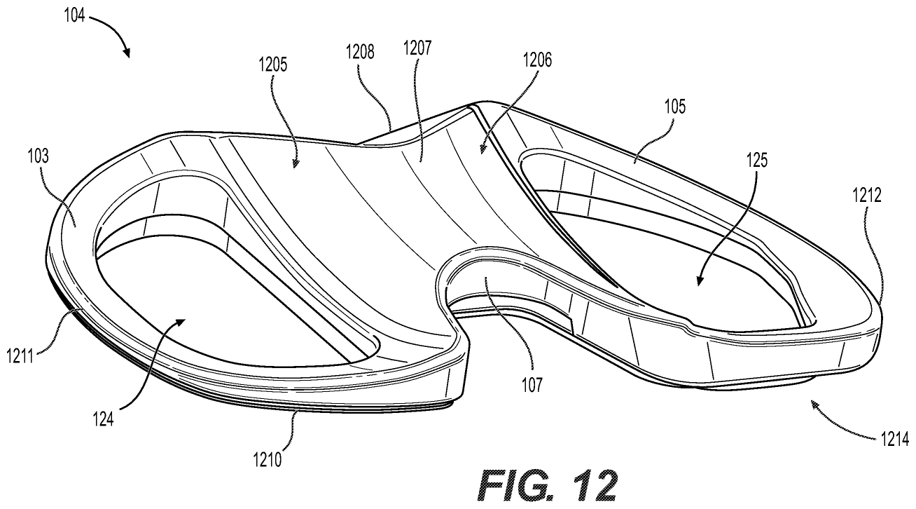

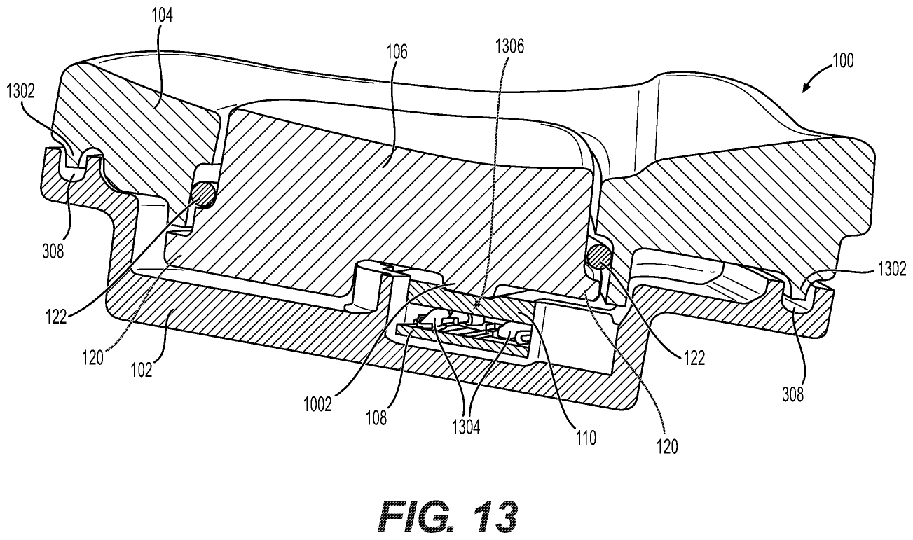

FIG. 12 is a perspective view of upper housing 104. Upper housing 104 may include lateral and medial portions 103, 105 connected by a joining portion 101. The lateral portion 103 and a portion of the joining portion 101 may make up a concave surface 1205. The medial portion 105 and a portion of the joining portion 101 similarly may make up a concave surface 1206. Thus, as shown, upper housing 104 may include two concave portions 1205, 1206 that meet at a peak 1207. Upper housing 104 may also include a sloped portion 1208 extending backward from the peak 1207 to the bottom surface 1210 of upper housing 104. Sloped portion 1208 may extend outward from the peak 1207 where the concave portions 1205, 1206 meet to the edges 1211, 1212 of the lateral and medial portions 103, 105. In some examples, upper housing 104 is configured to receive two plungers 106, 107. The lateral and medial portions 103, 105 of upper housing 104 may include lumens 124, 125 where plungers 106, 107 may be received. The concave surfaces 1205, 1206 may align with an upper surface 1001 of plungers 106, 107 so as to form a substantially even surface. Upper housing 104 may include a lateral lumen 124 and a medial lumen 125 configured to receive and house plungers 106, 107. Upper housing 104 may include a coupling portion 1302 (shown in FIG. 13) around the periphery of the upper housing bottom surface 1214. Coupling portion 1302 may be a ridge or raised portion around the periphery of the bottom surface 1214 of upper housing 104 that fits within receiving channel 308 of lower housing 102.

FIG. 13 is a partial cross-sectional view of implant 100 with lower housing 102 and upper housing 104 coupled together. As shown in FIG. 13, receiving channel 308 of lower housing 102 may receive coupling portion 1302 of upper housing 104 in order to couple lower housing 102 and upper housing 104 together. Receiving channel 308 may be configured to receive adhesive, such as a glue, before receiving coupling portion 1302 in order to hold lower hosing 102 and upper housing 104 together. Lower housing portion 102 and upper housing portion 104 may be hermetically sealed when coupled together. In some examples, O-ring 122, 123 may be held in place by retaining lip 120 and may create a seal between the plunger 106, 107 and upper housing 104. Thus, when coupled together, lower housing 102, upper housing 104, and plunger 106 may create a hermetically sealed interior. This hermetically sealed interior may prevent internal electronic components, such as PCB 108, from becoming damaged from external bodily material, such as bodily fluids, while also allowing plunger 106 to move relative to upper housing 104 and lower housing 102.

As shown in FIG. 13, bridge portion 1002 of plunger 106 may be in contact with sensors 110-112. Plunger 106 may be supported by only the bridge portions 1002 being in contact with at least three sensors 110-112. In some examples, the bridge portions 1002 and strain gauge sensors 110-112 may be equal in number. The bridge portions 1002 may be in contact with sensors 110-112 approximately at or near a central portion 1306 of sensors 110-112. As described herein, the strain gauge sensors 110-115 may define two polygons 801, 802, one in each of the lateral and medial portions 103, 105. Since the locations of the strain gauge sensors 110-115 in the polygons 801, 802 are known and the strain on each strain gauge sensor 110-115 in the polygons 801, 802 are known, the total load magnitude and position of the load applied to the upper housing and/or plungers may be calculated. For example, the load magnitude and load position may be calculated using triangulation or similar vector math. For a given time, each measured strain value from each of the strain gauge 110-115 readings are received at the remote system 200. A load magnitude and/or location may be determined using the measured strain values from each of the gauges 110-115.

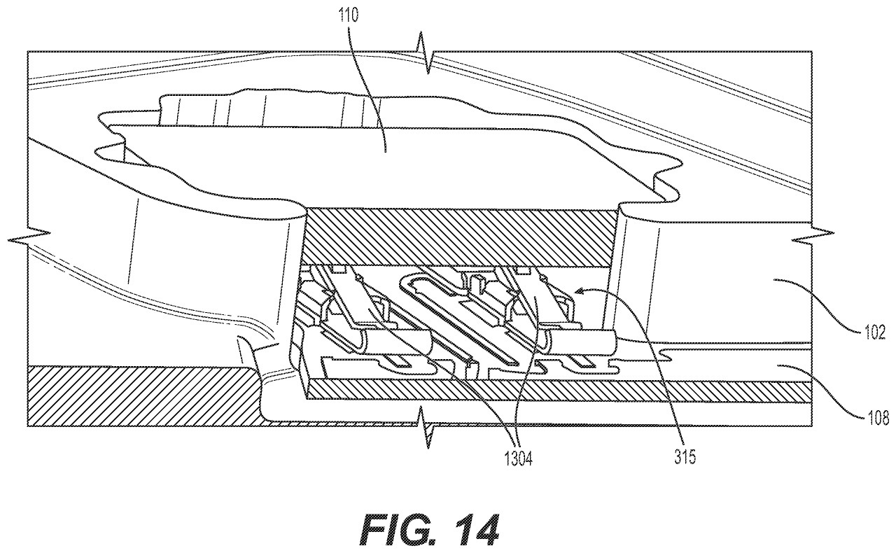

FIG. 14 shows an amplified, partial-cross-sectional view of sensor 110 mounted to PCB 108. Springs 1304 may be mounted to mounting portions 416 of PCB 108 and mounting points 712 of sensor 110-115. The springs 1304 may be flat springs, leaf springs, coil springs, or any connector with similar elastic properties. When the strain gauge sensor 110-115 is under load, the springs 1304 may allow each strain gauge sensor 110-115 to move or bend within each recess 310-315 of lower housing 102 when without putting excessive force on PCB 108. Excessive force may cause damage to the PCB 108 or medical implant 100.

A method of using the medical implant 100 may include placing the medical implant 100 within a patient's joint, for example a knee joint where a patient's femur and tibia meet, during a surgery, such as a total knee replacement surgery. The medical implant 100 may be a temporary insert used during the surgery and removed before implanting a permanent implant, or medical implant 100 may be a permanent implant. The medical implant 100 may gather information on force location and magnitude at the patient's joint. The medical implant 100 may communicate in real-time with a remote system 200. The remote system 200 may display real-time information on force location and magnitude at the joint, and may display historical information regarding force location and magnitude of force applied at the joint. In some examples, a surgeon may move one or more bones, such as tibia, relative to the joint and observe any changes in force location and magnitude on the remote system 200. The graphical user interface (GUI) of the remote system 200 may display the information as a number in text, graphically as a an image representation of the force, via color-coded regions of a virtual model of the joint (such as a knee joint), a combination thereof, or other display techniques known in the art. The surgeon may utilize the force data to make informed decisions, such as decisions related to the size and/or type of implant to use in the patient. Any of the components discussed herein may be used in any implant 100 to measure strain, load, range of motion, or other parameters related to a joint of the musculoskeletal system. For example, the devices of the present disclosure may be a tibial device, such as a tibial implant or tibial insert, used to measure at least load magnitude and load location placed on the tibial device.

As discussed herein, the tibial device 100 may include six internal strain gauge sensors 110. Three strain gauge sensors 110 may be placed in each of a lateral portion 103 and a medial portion 105, respectively, of the tibial device at predetermined locations around the perimeter of the device that define polygons 801, 802. When a load is placed on the upper housing 104, and specifically when the load is placed on a plunger 106, 107, the load placed on each plunger 106, 107 is transferred to the three strain gauge sensors 110-115 that support the plunger 106, 107. As the three strain gauge sensors 110-115 are supporting each plunger 106, 107 as the only contact points below the applied load, the strain gauge sensors 110-115 may be used to calculate the total load magnitude placed on the plunger 106, 107. Further, the different loads measured by each strain gauge sensor 110-115 may be used, along with the known predetermined location of each strain gauge sensor 110-115, to determine the location of the load on each plunger 106, 107.

Once the applied strain has been measured by strain gauge sensors 110-115, the measured strain may be used to calculate the applied load on implant 100 internally using the electronic components 422, 424 in real time. In some examples, the measured strain information may be transmitted from implant 100 to an external device, such as a computing device, where the load parameters are then calculated. An external device that receives strain measurements or load parameters may include a display with a graphical user interface (GUI). The load magnitude and location may be displayed on the GUI of the external device. For example, the GUI may include a graphical representation of the tibial device and/or a graphical representation of the tibial device positioned within a knee joint including a representation of a tibia and/or a femur. The graphical representation of a location where the load is measured on the tibial device may include a light that changes color to indicate the magnitude of the load. In another example, the GUI may include a text representation (readout) of the location and magnitude of loads on the tibial device.

Surgeons and other medical personnel may utilize load information displayed on the GUI. In one example, when the tibial device is a tibial trial insert, a surgeon may use load information displayed on a GUI to make decisions on surgery, for example where to cut bone or how to align the tibia and femur. The strain gauge sensors 700 described herein may have advantages over other types of load sensors, for example capacitive sensors or other sensors using strain gauges. These strain gauge sensors 700 may be more accurate than other sensors because they have reduced drift, reduced temperature sensitivity shift, reduced noise, reduced hysteresis, reduced residual load, improved sensitivity, and improved temperature stability. Further, these strain gauge sensors 700 may be easy and cheap to manufacture relative to other strain sensors. The strain gauge sensors 700 as described herein are also simple to mount to PCB 108 using springs or surface mounting technology (SMT). Overall, strain gauge sensor 700 is uniquely configured to support accurate, repeatable, and cost efficient force, pressure, or load sensing for medical applications.

It will be apparent to those skilled in the art that various modifications and variations can be made to the disclosed system without departing from the scope of the disclosure. Other embodiments of the system will be apparent to those skilled in the art from consideration of the specification and practice of the counterweight system disclosed herein. It is intended that the specification and examples be considered as exemplary only, with a true scope of the disclosure being indicated by the following claims and their equivalents.

What is claimed is:

1. A medical device, comprising:
a lower housing;
a circuit board within the lower housing;
a first plurality of strain gauge sensors mounted to a medial portion of the circuit board;
a second plurality of strain gauge sensors mounted to a lateral portion of the circuit board;
an upper housing coupled to the lower housing;
a first plunger received by the upper housing and in contact with each of the first plurality of strain gauge sensors; and
a second plunger received by the upper housing and in contact with each of the second plurality of strain gauge sensors.

2. The medical device of claim 1, wherein the first plunger comprises a first plurality of bridge portions;
wherein the second plunger comprises a second plurality of bridge portions;
wherein each bridge portion of the first plurality of bridge portions is in contact with a strain gauge sensor of the first plurality of strain gauge sensors; and
wherein each bridge portion of the second plurality of bridge portions is in contact with a strain gauge sensor of the second plurality of strain gauge sensors.

3. The medical device of claim 2, wherein the first plunger is configured such that a first force applied to a top side of the first plunger is applied across the first plurality of strain gauge sensors; and
wherein the second plunger is configured such that a second force applied to a top side of the second plunger is applied across the second plurality of strain gauge sensors.

4. The medical device of claim 3, further comprising:
wherein the first plurality of strain gauge sensors are positioned to define a medial polygon;
wherein vertices of the medial polygon are at center points of each of the first plurality of strain gauge sensors;
wherein the second plurality of strain gauge sensors are positioned to define a lateral polygon; and
wherein vertices of the lateral polygon are at center points of each of the second plurality of strain gauge sensors.

5. The medical device of claim 4, further comprising:
wherein the first plurality of strain gauge sensors comprise a first strain gauge sensor mounted at a first mounting area, a second strain gauge sensor mounted at a second mounting area, and a third strain gauge sensor mounted at a third mounting area;
wherein the first mounting area, the second mounting area, and the third mounting area are positioned around the perimeter of the medial portion of the circuit board and protrude outwardly from a central portion of the medial portion;
wherein the second plurality of strain gauge sensors comprise a fourth strain gauge sensor mounted at a fourth mounting area, a fifth strain gauge sensor mounted at a fifth mounting area, and a sixth strain gauge sensor mounted at a sixth mounting area; and
wherein the fourth mounting area, the fifth mounting area, and the sixth mounting area are positioned around the perimeter of the lateral portion of the circuit board and protrude outwardly from a central portion of the lateral portion.

6. The medical device of claim 5, further comprising:
wherein the first strain gauge sensor is mounted such that a first reference axis of the first strain gauge sensor forms an angle of 0-15 degrees with a first central longitudinal axis of the medial portion, wherein the first reference axis extends perpendicular to a second central longitudinal axis of the first strain gauge sensor and across a first width of the first strain gauge sensor;
wherein the second strain gauge sensor is mounted such that a second reference axis of the second strain gauge sensor forms an angle of 0-40 degrees with the first central longitudinal axis, wherein the second reference axis extends perpendicular to a third central longitudinal axis of the second strain gauge sensor and across a second width of the second strain gauge sensor; and
wherein the third strain gauge sensor is mounted such that a third reference axis of the third strain gauge sensor forms an angle of 0-90 degrees with the first central longitudinal axis, wherein the third reference axis extends perpendicular to a fourth central longitudinal axis of the third strain gauge sensor and across a third width of the third strain gauge sensor.

7. The medical device of claim 6, wherein the first strain gauge sensor, the second strain gauge sensor, and the third strain gauge sensor are angled relative to each other.

8. The medical device of claim 6, wherein the first plunger includes a first o-ring configured to form a seal with the upper housing; and the second plunger includes a second o-ring configured to form a seal with the upper housing.

9. The medical device of claim 6, wherein each strain gauge sensor of the first plurality of strain gauge sensors is coupled to a separate mounting portion of the first plurality of mounting portions, and each strain gauge sensor of the second plurality of strain gauge sensors is coupled to a separate mounting portion of the second plurality of mounting portions.

10. The medical device of claim 6, wherein the first plurality of strain gauge sensors and the second plurality of strain gauge sensors are mounted to the circuit board with springs.

11. The medical device of claim 1, wherein each of the first plurality of strain gauge sensors and each of the second plurality of strain gauge sensors include a plurality of strain sensors.

12. A method of determining a load magnitude and load location at a joint using a medical device, the method comprising:
measuring strain at a first plurality of strain gauge sensors and a second plurality of strain gauge sensors;
determining at least one load magnitude and at least one load location based on the measured strain; wherein the medical device comprises:
a lower housing;
a circuit board within the lower housing;
the first plurality of strain gauge sensors mounted to a medial portion of the circuit board;
the second plurality of strain gauge sensors mounted to a lateral portion of the circuit board;
an upper housing coupled to the lower housing;
a first plunger received by the upper housing and adjacent to the first plurality of strain gauge sensors; and
a second plunger received by the upper housing and adjacent to the second plurality of strain gauge sensors.

13. The method of claim 12, wherein the first plunger further comprises a first plurality of bridge portions;
wherein the second plunger further comprises a second plurality of bridge portions;
wherein each bridge portion of the first plurality of bridge portions is in contact with a strain gauge sensor of the first plurality of strain gauge sensors; and
wherein each bridge portion of the second plurality of bridge portions is in contact with a strain gauge sensor of the second plurality of strain gauge sensors.

14. The method of claim 13, wherein the first plurality of strain gauge sensors are positioned to define a medial polygon;
wherein vertices of the medial polygon are at center points of each of the first plurality of strain gauge sensors;
wherein the second plurality of strain gauge sensors are positioned to define a lateral polygon between them; and
wherein vertices of the lateral polygon are at center points of each of the second plurality of strain gauge sensors.

15. The method claim 14, further comprising:
measuring a first load applied to the first plunger by measuring a first strain on each of the strain gauge sensors that define the medial polygon; and
measuring a second load applied to the second plunger by measuring a second strain on each of the strain gauge sensors that define the lateral polygon.

16. The method of claim 15, further comprising:
determining a medial load magnitude and a medial load location on the first plunger from the first strain; and
determining a lateral load magnitude and lateral load location on the second plunger from the second strain.

17. The method of claim 16, further comprising:
displaying the medial load magnitude, medial load location, lateral load magnitude, and lateral load location on a graphical user interface (GUI).

18. A medical device, comprising:
a lower housing;
a circuit board within the lower housing comprising a medial portion and a lateral portion;
a first plurality of strain gauge sensors comprising at least three strain gauge sensors mounted to the medial portion;
a second plurality of strain gauge sensors comprising at least three strain gauge sensors mounted to the lateral portion;
an upper housing coupled to the lower housing, the upper housing comprising a lateral lumen and a medial lumen;
a medial plunger configured to be received in the medial lumen;
a lateral plunger configured to be received in the lateral lumen;
wherein the first plurality of strain gauge sensors are in contact with a first surface of the medial plunger;
wherein the second plurality of strain gauge sensors are in contact with a second surface of the lateral plunger; and
wherein the first plurality of strain gauge sensors and the second plurality of strain gauge sensors are mounted to the circuit board via springs.

19. The medical device of claim 18,
wherein the medial plunger further comprises a first plurality of bridge portions;
wherein the lateral plunger further comprises a second plurality of bridge portions;
wherein each bridge portion of the first plurality of bridge portions is in contact with a strain gauge sensor of the first plurality of strain gauge sensors; and
wherein each bridge portion of the second plurality of bridge portions is in contact with a strain gauge sensor of the second plurality of strain gauge sensors.

20. The medical device of claim 19, wherein the medial plunger is configured to distribute force applied to a top side of the medial plunger across the first plurality of strain gauge sensors; and
wherein the lateral plunger is configured to distribute force applied to a top side of the lateral plunger across the second plurality of strain gauge sensors.
